(19) United States
(12) Patent Application Publication
(10) Pub. No.: US 2021/0338543 A1
(43) Pub. Date: Nov. 4, 2021

(54) WATER-IN-OIL EMULSIONS CONTAINING LATEX AND SILOXYSILICATE RESIN

(71) Applicant: L'ORÉAL, Paris (FR)

(72) Inventors: Aline GUIMONT, South Orange, NJ (US); Geoffrey WHITE, Old Bridge, NJ (US)

(73) Assignee: L'ORÉAL, Paris (FR)

(21) Appl. No.: 16/863,040

(22) Filed: Apr. 30, 2020

(51) Int. Cl. A61K 8/06; A61Q 1/10; A61K 8/92; A61K 8/891; A61K 8/81

(52) U.S. Cl. CPC A61K 8/064; A61Q 1/10; A61K 8/92; A61K 2800/10; A61K 8/8152; A61K 2800/43; A61K 8/891

(57) ABSTRACT

The invention relates to water-in-oil (w/o) emulsion compositions for hair, eyelashes and/or eyebrows including at least one latex containing polymer containing at least one styrene group and at least one siloxysilicate resin, as well as to methods of making and using such emulsions. Preferably, the composition is a composition for eyelashes, for example a mascara composition further including at least one colorant.

### FIELD OF THE INVENTION

The present invention relates to water-in-oil (w/o) emulsions (compositions) comprising at least one latex comprising polymer comprising at least one styrene group and at least one siloxysilicate resin. The compositions have beneficial cosmetic properties including good or improved wear, sebum resistance and/or water resistance properties. Significantly, the compositions also have good or improved removal properties.

### DISCUSSION OF THE BACKGROUND

Mascara compositions are commonly used to enhance the appearance of eyelashes. Conventional mascara compositions generally use waxes to form crystalline network structures to enhance curl, volume, length, thickness, and/or colors to eyelashes. However, conventional mascara compositions including waxes tend to become less resistant to oil and/or sebum, causing smearing, flaking, and/or color transferring after wearing for a certain amount of time.

Mascaras are in particular prepared on the basis of two types of formulations: aqueous mascaras referred to as cream mascaras, in the form of a dispersion of waxes in water, and anhydrous or low-water-content mascaras, referred to as water-resistant mascaras (referred to as “waterproof”), in the form of dispersions of waxes in organic solvents.

The aqueous mascaras mainly contain a surfactant system, for example based on triethanolamine stearate, which makes it possible to obtain a stable dispersion of particles of wax agglomerated in an aqueous phase. This system plays an important part in the obtaining of such a dispersion, in particular at the interface in the interactions between particles of wax.

However, the mascaras described above have the disadvantages of, among other things, being dry, having poor flexibility and/or having poor consistency. Also, consumers have difficulty removing such mascaras from eyelashes.

WO 2019/122102 is directed to a direct emulsion for use on lips containing at least 10% silicone resin.

WO 2014/046883 is directed to a slurry powder cosmetic composition containing particulates coated with a binder composition, a silicone gel composition, and the evaporation residue of an alcohol based composition and a method for making the composition.

WO 2010/149493 is directed to composition for making up and/or caring for keratin materials, comprising at least one fatty phase, at least one silicone resin and at least one styrene/acrylate copolymer.

WO 2014/143757 is directed to compositions containing a silicone resin emulsion containing 0.5 wt % to 95 wt % of a silicone resin or pressure sensitive adhesive (PSA), 0.1 to 90 wt % of an ethylene oxide/propylene oxide block copolymer, and sufficient amount of water to sum to 100 weight percent.

U.S. Pat. No. 10,441,527 is directed to fluid compositions containing multiple components, the first of which can include a copolymer which is a cross-linked siloxane (e.g. a cross-linked aminosiloxane), a silicone polyether copolymer (e.g. an (AB)n silicone polyether copolymer), and/or a saccharide siloxane copolymer, and the second of which can include organopolysiloxane resin and/or an acrylate copolymer.

FR 2968983 is directed to compositions fluid anhydrous compositions containing at least one organopolysiloxane elastomer powder coated with silicone resin; at least one organopolysiloxane elastomer vehicle in a first oil; at least one wax; at least one organic lacquer; and at least a second oil.

US 2006/0078578 is directed to film forming compositions containing dispersed non-quaternary montmorillonite mineral and a film forming component.

There is a need to develop a cosmetic composition, in particular for making up the eyelashes, making it possible to obtain a good wearing, water- and sebum-resistant product, which is preferably easy to remove after use.

### SUMMARY OF THE INVENTION

The present invention relates to water-in-oil (w/o) emulsion compositions for hair, eyelashes and/or eyebrows comprising at least one latex comprising polymer comprising at least one styrene group, wherein the composition comprises from about 2% to about 6% by weight with respect to the total weight of the composition of the polymer comprising at least one styrene group, and at least one siloxysilicate resin. Preferably, the composition is a composition for eyelashes. Most preferably, the composition is a mascara composition further comprising at least one colorant.

The present invention relates to water-in-oil (w/o) emulsion compositions for hair, eyelashes and/or eyebrows comprising at least one latex comprising polymer comprising at least one styrene group, wherein the composition comprises from about 2% to about 6% by weight with respect to the total weight of the composition of the polymer comprising at least one styrene group, at least one siloxysilicate resin, and at least one wax. Preferably, the composition is a composition for eyelashes. Most preferably, the composition is a mascara composition further comprising at least one colorant.

The present invention also relates to methods of treating, caring for and/or making up hair, eyelashes, and/or eyebrows by applying compositions of the present invention to hair, eyelashes, and/or eyebrows in an amount sufficient to treat, care for and/or make up the hair, eyelashes, and/or eyebrows. Preferably, the composition is a composition for eyelashes. Most preferably, the composition is a mascara composition further comprising at least one colorant.

The present invention also relates to methods of improving the removal properties (from hair, eyebrows and/or eyelashes) of a water-in-oil (w/o) emulsion composition comprising at least one siloxysilicate resin, and optionally at least one wax, by adding at least one latex comprising polymer comprising at least one styrene group in an amount sufficient to add from about 2% to about 6% by weight with respect to the total weight of the composition of the polymer comprising at least one styrene group to the composition. Preferably, the composition is a composition for eyelashes. Most preferably, the composition is a mascara composition further comprising at least one colorant.

The present invention also relates to methods of making a water-in-oil (w/o) emulsion composition for hair, eyelashes and/or eyebrows comprising combining at least one latex comprising polymer comprising at least one styrene group in an amount sufficient to add from about 2% to about 6% by weight with respect to the total weight of the composition of the polymer comprising at least one styrene group to the composition, at least one siloxysilicate resin, and optionally at least one wax, to form the composition. Preferably, the composition is a composition for eyelashes. Most preferably, the composition is a mascara composition further comprising at least one colorant.

### DETAILED DESCRIPTION OF THE INVENTION

As used herein, the expression “at least one” means one or more and thus includes individual components as well as mixtures/combinations.

Other than in the operating examples, or where otherwise indicated, all numbers expressing quantities of ingredients and/or reaction conditions are to be understood as being modified in all instances by the term “about,” meaning within 10% of the indicated number.

“Film former” or “film forming agent” as used herein means a polymer or resin that leaves a film on the substrate to which it is applied, for example, after a solvent accompanying the film former has evaporated, absorbed into and/or dissipated on the substrate.

“Transfer resistance” as used herein refers to the quality exhibited by compositions that are not readily removed by contact with another material, such as, for example, an item of clothing or the skin, for example. “Smudge resistance” is a type of “transfer resistance”—in the context of mascara compositions, smudges occur when eyelashes contact skin around the eye area and mascara transfers to the contacted skin area. Transfer resistance may be evaluated by any method known in the art for evaluating such. For example, transfer resistance of a composition may be evaluated by a test which may involve application of the composition to human keratin material such as hair, eyelashes or eyebrows followed by rubbing a material, for example, a sheet of paper, against the hair, eyelashes or eyebrows after expiration of a certain amount of time following application, such as 2 minutes after application. Similarly, transfer resistance of a composition may be evaluated by the amount of product transferred from a wearer to any other substrate, such as transfer from the hair, eyelashes or eyebrows of an individual to an item of clothing or skin after the expiration of a certain amount of time following application of the composition to the hair, eyelashes or eyebrows. The amount of composition transferred to the substrate (e.g., collar, skin or paper) may then be evaluated and compared. For example, a composition may be transfer resistant if a majority of the product is left on the wearer's hair, eyelashes or eyebrows. Further, the amount transferred may be compared with that transferred by other compositions, such as commercially available compositions. In a preferred embodiment of the present invention, little or no composition is transferred to the substrate from the hair, eyelashes or eyebrows.

“Long wear” compositions as used herein, refers to compositions where color remains the same or substantially the same as at the time of application, as viewed by the naked eye, after an extended period of time. Long wear properties may be evaluated by any method known in the art for evaluating such properties. For example, long wear may be evaluated by a test involving the application of a composition to human hair, eyelashes or eyebrows and evaluating the color of the composition after an extended period of time. For example, the color of a composition may be evaluated immediately following application to hair, eyelashes or eyebrows and these characteristics may then be re-evaluated and compared after a certain amount of time. Further, these characteristics may be evaluated with respect to other compositions, such as commercially available compositions.

“Waterproof” as used herein refers to the ability to repel water and permanence with respect to water. Waterproof properties may be evaluated by any method known in the art for evaluating such properties. For example, a mascara composition may be applied to false eyelashes, which may then be placed in water for a certain amount of time, such as, for example, 20 minutes. Upon expiration of the pre-ascertained amount of time, the false eyelashes may be removed from the water and passed over a material, such as, for example, a sheet of paper. The extent of residue left on the material may then be evaluated and compared with other compositions, such as, for example, commercially available compositions. For example, a composition may be waterproof if a majority of the product is left on the wearer, e.g., eyelashes. In a preferred embodiment of the present invention, little or no composition is transferred from the wearer.

“Substituted” as used herein, means comprising at least one substituent. Non-limiting examples of substituents include atoms, such as oxygen atoms and nitrogen atoms, as well as functional groups, such as hydroxyl groups, ether groups, alkoxy groups, acyloxyalkyl groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, and polysiloxane groups. The substituent(s) may be further substituted.

“Volatile”, as used herein, means having a flash point of less than about 100° C.

“Non-volatile”, as used herein, means having a flash point of greater than about 100° C.

“Removal properties,” as used herein, refers to removing compositions from hair, eyebrows and/or eyelashes. “Easy removal properties” for a removal process refer to processes which are not as time-consuming and/or which do not substantially damage hair, eyebrows and/or eyelashes. Removal properties can be evaluated, for example, by comparing removability of a composition using a removal solvent (for example, a water-based composition containing soap/surfactant or an oil-based composition) with removability of other compositions (including commercially-available compositions) using the same solvent. For example, compositions being evaluated can be applied to hair, eyebrows and/or eyelashes (real or artificial), and allowed to dry for a designated period of time (for example, 15 minutes). Then, the number of strokes it takes to completely remove the composition from the hair, eyebrows and/or eyelashes using a removal solvent and cotton pad can be counted and compared with other compositions.

The compositions and methods of the present invention can comprise, consist of, or consist essentially of the essential elements and limitations of the invention described herein, as well as any additional or optional ingredients, components, or limitations described herein or otherwise useful. For example, the film-forming component of the emulsion composition can “consist essentially of” or “consist of” latex(es) comprising polymer comprising at least one styrene group and siloxysilicate resin(s).

The “film-forming component” of the emulsion composition of the present invention comprises at least one latex comprising polymer comprising at least one styrene group and at least one siloxysilicate resin. It may optionally further contain one or more additional film-forming agents. Preferably, the film-forming component contains a majority (greater than 50% by weight) of polymer comprising at least one styrene group and siloxysilicate resin, preferably greater than 60% by weight, preferably greater than 70% by weight, preferably greater than 80% by weight, preferably greater than 90% by weight, and may contain up to 100% by weight of these film-forming agents, all percentages being based on total weight of the two types of film-forming agents with respect to the total weight of the film-forming component. All ranges and subranges within the percentages set forth above are included herein such as, for example, 10%-100% by weight, 25%-90% by weight, 50%-85% by weight, etc.

The “film-forming component” of the emulsion composition of the present invention preferably comprises from 1% to 15% by weight with respect to the total weight of the composition, preferably 2% to 10% by weight with respect to the total weight of the composition, and preferably from 3% to 8% by weight with respect to the total weight of the composition, including all ranges and subranges therebetween.

For purposes of the emulsion compositions and components of the present invention which “consist essentially of” identified ingredients, the “basic and novel properties” of such compositions and component is removal properties.

For purposes of the methods of the present invention directed to improving removal properties of an emulsion composition which “consists essentially of” identified ingredients, the “basic and novel property” of such methods is improving removal properties of a composition.

As indicated above, the compositions of the present invention comprise “latex,” which is a colloidal dispersion of polymer particles in an aqueous liquid phase. “Latex” is generally obtained by suspension or emulsion polymerization or copolymerization of monomers according to processes that are well known to those of ordinary skill in the art. Such monomers may be chosen in particular from styrene, butadiene, acrylonitrile, chloroprene, vinyl acetate, urethanes, isoprene, isobutylene, and acrylic or methacrylic acid, maleic acid, crotonic acid or itaconic acid or esters or amides thereof.

Latex Comprising Polymer Comprising at Least One Styrene Group

In accordance with the present invention, water-in-oil emulsion compositions comprising at least one latex comprising polymer comprising at least one styrene group, wherein the composition comprises from about 2% to about 6% by weight with respect to the total weight of the composition of the polymer comprising at least one styrene group, are provided. According to preferred embodiments, the latex is an aqueous dispersion of styrene/acrylate copolymer.

In particular, the latex preferably comprises a polymer chosen from copolymers resulting from the polymerization of at least one styrene monomer and at least one C₁-C₁₈ alkyl (meth)acrylate monomer, preferably C₁-C₁₂ alkyl (meth)acrylate, and preferably C₁-C₁₀ alkyl (meth)acrylate. The C₁-C₁₈ alkyl (meth)acrylate monomer is preferably chosen from methyl acrylate, methyl methacrylate, ethyl acrylate, propyl acrylate, butyl acrylate, butyl methacrylate, hexyl acrylate, octyl acrylate, 2-ethylhexyl acrylate, lauryl (meth)acrylate and stearyl (meth)acrylate. As styrene monomer that may be used in the invention, examples that may be mentioned include styrene and α-methylstyrene.

According to preferred embodiments, the polymer comprising at least one styrene group is at least one high molecular weight polymer comprising at least one styrene group. As used herein, “high molecular weight” means more than 100,000 Da weight average molecular weight, preferably more than 120,000 Da, preferably more than 135,000 Da, and preferably more than 150,000 Da, including all ranges and subranges therebetween such as, for example, 100,000 Da-500,000 Da, 120,000 Da to 600,000 Da, 150,000 Da to 200,000 Da, etc.

Any styrene acrylate copolymer or derivative thereof may be suitable for use in the compositions of the present invention. The latex comprising polymer comprising at least one styrene group preferably has a glass transition temperature (Tg) ranging from about −15° C. to about 90° C., such as from about 0° C. to about 50° C., such as from about 5° C. to about 25° C., such as from about 10° C. to about 20° C., including all ranges and subranges therebetween. For example, a copolymer comprising (1) styrene and ammonium acrylates, (2) acrylates, and/or (3) acrylic groups are suitable. A particularly preferred copolymer is a styrene/acrylates/ammonium methacrylate copolymer or a styrene acrylic copolymer, commercial examples of which include SYNTRAN 5620 and SYNTRAN 5760 (with or without paraben), by Interpolymer Corporation; JONCRYL 77, by BASF Performance Chemicals; and RHOPLEX P376, by Dow Chemical Company.

Preferably, the latex comprising polymer comprising at least one styrene group is present in the compositions of the present invention in an amount ranging from about 5% to about 15%, more preferably from about 5% to about 12%, and preferably from about 5% to about 8%, by weight, based on the total weight of the composition, including all ranges and subranges in between. The latex comprising polymer comprising at least one styrene group comprises (1) polymer comprising at least one styrene group; and (2) liquid phase carrier for the polymer.

According to preferred embodiments, the polymer comprising at least one styrene group is present in an amount ranging from about 2% to about 6%, preferably from about 2.25% to about 5.5%, and preferably from about 2.5% to about 5%, by weight, based on the total weight of the composition, including all ranges and subranges in between.

Surprisingly, it has been found that water-in-oil (w/o) emulsion compositions comprising at least one siloxysilicate resin, and optionally at least one wax, containing the polymer comprising at least one styrene group in amounts both below and above the identified concentration range have inferior removal properties as compared to such emulsions containing the polymer comprising at least one styrene group in amounts falling within the identified concentration range. Also surprisingly, it has been found that such increase in removability properties within the identified concentration range is generally independent of the amount of siloxysilicate resin present in the emulsions.

Siloxysilicate Resin

According to the present invention, compositions comprising at least one siloxysilicate resin are provided. Siloxysilicate resins generally have the formula;

[(R)₃SiO]ₓ(SiO_{4/2})_{y}

Where R is an alkyl group preferably comprising 1 to 8 carbon atoms. One non-limiting example of a siloxysilicate in accordance with the present invention is trimethylsiloxysilicate, which may be represented by the following formula:

[(CH₃)₃SiO]ₓ(SiO_{4/2})_{y}.

In the above formulas, x and y preferably range between numbers such as, 10 and 150, 25 and 125, 35 and 100, and 50 to 80, for example.

Preferred siloxysilicate resins are commercially available from General Electric, Dow Corning, Wacker, Milliken, Siltech, Grant Industries, Momentive and Shin-Etsu Silicones under the tradename Resin MQ®.

The at least one siloxysilicate resin is preferably present in the compositions of the present invention in an amount of less than 15% by weight, preferably less than 10% by weight, including all ranges and subranges therebetween such as, for example, 1% to 15% by weight, 3% to 12% by weight, 1% to 10% by weight, 3% to 8% by weight, etc., all weights being based on the weight of the composition as a whole.

Although not wishing to be bound by any theories, generally speaking compositions comprising at least one latex comprising polymer comprising at least one styrene group and at least one siloxysilicate resin have good or improved removal properties while maintaining beneficial cosmetic properties including good or improved wear, sebum resistance and/or water resistance properties. Given that both the latex and the siloxysilicate resin would be expected to increase long-wear properties of a composition, it would be expected that increasing concentrations of both in a composition would result in a corresponding increase in removal difficulty. However, contrary to this expectation, it was surprisingly discovered that, at concentrations of from about 2% to about 6%, such amounts of polymer comprising at least one styrene group actually improved removal properties of the compositions, including in mascara compositions containing low wax levels, whereas concentration levels on either side of this range resulted in emulsions having inferior removal properties.

According to particularly preferred embodiments, the combined amount of siloxysilicate resin and of polymer comprising at least one styrene group present ranges from about 5% to 25% by weight, preferably from about 7.5% to about 20%, preferably about 10% to about 15% by weight based on the total weight of the composition, including all ranges and subranges within these ranges. Also, the weight ratio of siloxysilicate resin to polymer comprising at least one styrene group present preferably ranges from 1:1 to about 5:1, preferably from about 2:1 to about 4:1.

Water

According to the present invention, compositions comprising water are provided. Compositions of the present invention comprise sufficient water to form a water-in-oil emulsion, preferably from about 5% to about 80% water, more preferably from about 10% to about 50% water, and more preferably from about 15% to about 30% water by weight with respect to the total weight of the composition, including all ranges and subranges therebetween.

Oil

According to the present invention, compositions comprising at least one oil are provided. Compositions of the present invention comprise sufficient oil to form a water-in-oil emulsion, preferably from about 5% to about 80% oil, more preferably from about 10% to about 50% oil, and more preferably from about 15% to about 30% oil by weight with respect to the total weight of the composition, including all ranges and subranges therebetween.

Suitable oils include volatile and/or non-volatile oils. Such oils can be any acceptable oil including but not limited to silicone oils and/or hydrocarbon oils.

According to certain embodiments, the oil carrier comprises one or more volatile silicone oils. Examples of such volatile silicone oils include linear or cyclic silicone oils having a viscosity at room temperature less than or equal to 6 cSt and having from 2 to 7 silicon atoms, these silicones being optionally substituted with alkyl or alkoxy groups of 1 to 10 carbon atoms. Specific oils that may be used in the invention include octamethyltetrasiloxane, decamethylcyclopentasiloxane, dodecamethylcyclohexasiloxane, heptamethyloctyltrisiloxane, hexamethyldisiloxane, decamethyltetrasiloxane, dodecamethylpentasiloxane and their mixtures. Other volatile oils which may be used include KF 96A of 6 cSt viscosity, a commercial product from Shin Etsu having a flash point of 94° C. Preferably, the volatile silicone oils have a flash point of at least 40° C.

Non-limiting examples of volatile silicone oils are listed in Table 1 below.

TABLE 1

| | Flash Point | Viscosity |
|---|---|---|
| Compound | (° C.) | (cSt) |
| Octyltrimethicone | 93 | 1.2 |
| Hexyltrimethicone | 79 | 1.2 |
| Decamethylcyclopentasiloxane | 72 | 4.2 |
| (cyclopentasiloxane or D5) | | |
| Octamethylcyclotetrasiloxane | 55 | 2.5 |
| (cyclotetradimethylsiloxane or D4) | | |
| Dodecamethylcyclohexasiloxane (D6) | 93 | 7 |
| Decamethyltetrasiloxane(L4) | 63 | 1.7 |
| KF-96 A from Shin Etsu | 94 | 6 |
| PDMS (polydimethylsiloxane) DC 200 | 56 | 1.5 |
| (1.5 cSt) from Dow Corning | | |
| PDMS DC 200 (2 cSt) from Dow Corning | 87 | 2 |

Further, a volatile linear silicone oil may be employed in the present invention. Suitable volatile linear silicone oils include those described in U.S. Pat. No. 6,338,839 and WO03/042221, the contents of which are incorporated herein by reference. In one embodiment the volatile linear silicone oil is decamethyltetrasiloxane. In another embodiment, the decamethyltetrasiloxane is further combined with another solvent that is more volatile than decamethyltetrasiloxane.

According to other embodiments, the oil carrier comprises one or more non-silicone volatile oils and may be selected from volatile hydrocarbon oils, volatile esters and volatile ethers. Examples of such volatile non-silicone oils include, but are not limited to, volatile hydrocarbon oils having from 8 to 16 carbon atoms and their mixtures and in particular branched C₈ to C₁₆ alkanes such as C₈ to C₁₆ isoalkanes (also known as isoparaffins), isododecane, isodecane, and for example, the oils sold under the trade names of Isopar or Permethyl. Preferably, the volatile non-silicone oils have a flash point of at least 40° C.

Non-limiting examples of volatile non-silicone volatile oils are given in Table 2 below.

TABLE 2

| Compound | Flash Point (° C.) |
|---|---|
| Isododecane | 43 |
| Propylene glycol n-butyl ether | 60 |
| Ethyl 3-ethoxypropionate | 58 |
| Propylene glycol methylether acetate | 46 |
| Isopar L (isoparaffin C₁₁-C₁₃) | 62 |
| Isopar H (isoparaffin C₁₁-C₁₂) | 56 |

The volatility of the solvents/oils can be determined using the evaporation speed as set forth in U.S. Pat. No. 6,338,839, the contents of which are incorporated by reference herein.

According to other embodiments of the present invention, the oil carrier comprises at least one non-volatile oil. Examples of non-volatile oils that may be used in the present invention include, but are not limited to, polar oils such as:
hydrocarbon-based plant oils with a high triglyceride content consisting of fatty acid esters of glycerol, the fatty acids of which may have varied chain lengths, these chains possibly being linear or branched, and saturated or unsaturated; these oils are especially wheat germ oil, corn oil, sunflower oil, karite butter, castor oil, sweet almond oil, macadamia oil, apricot oil, soybean oil, rapeseed oil, cottonseed oil, alfalfa oil, poppy oil, pumpkin oil, sesame seed oil, marrow oil, avocado oil, hazelnut oil, grape seed oil, blackcurrant seed oil, evening primrose oil, millet oil, barley oil, quinoa oil, olive oil, rye oil, safflower oil, candlenut oil, passion flower oil or musk rose oil; or caprylic/capric acid triglycerides, for instance those sold by the company Stearineries Dubois or those sold under the names Miglyol 810, 812 and 818 by the company Dynamit Nobel;
synthetic oils or esters of formula R₅COOR₆ in which R₅ represents a linear or branched higher fatty acid residue containing from 1 to 40 carbon atoms, including from 7 to 19 carbon atoms, and R₆ represents a branched hydrocarbon-based chain containing from 1 to 40 carbon atoms, including from 3 to 20 carbon atoms, with R₆+R₇≥10, such as, for example, Purcellin oil (cetostearyl octanoate), isononyl isononanoate, C₁₂ to C₁₅ alkyl benzoate, isopropyl myristate, 2-ethylhexyl palmitate, and octanoates, decanoates or ricinoleates of alcohols or of polyalcohols; hydroxylated esters, for instance isostearyl lactate or diisostearyl malate; and pentaerythritol esters;
synthetic ethers containing from 10 to 40 carbon atoms;
C₈ to C₂₆ fatty alcohols, for instance oleyl alcohol, cetyl alcohol, stearyl alcohol, and cetearyl alcohol; and
mixtures thereof.

Further, examples of non-volatile oils that may be used in the present invention include, but are not limited to, non-polar oils such as branched and unbranched hydrocarbons and hydrocarbon waxes including polyolefins, in particular Vaseline (petrolatum), paraffin oil, squalane, squalene, hydrogenated polyisobutene, hydrogenated polydecene, polybutene, mineral oil, pentahydrosqualene, and mixtures thereof.

Compositions of the present invention can optionally further comprise any additive usually used in the field(s) under consideration. For example, waxes, dispersants, antioxidants, sunscreens, preserving agents, fragrances, fibers, fillers, neutralizing agents, cosmetic and dermatological active agents such as, for example, emollients, moisturizers, vitamins, essential fatty acids, surfactants, silicone elastomers, pasty compounds, viscosity increasing agents, for example, liposoluble/lipodispersible polymers, additional film forming agents, colorants, and mixtures thereof can be added. A non-exhaustive listing of such ingredients can be found in U.S. patent application publication nos. 2004/0170586 and 2009/0142289, the entire contents of which are hereby incorporated by reference. Further examples of suitable additional components can be found in the other references which have been incorporated by reference in this application. Still further examples of such additional ingredients may be found in the International Cosmetic Ingredient Dictionary and Handbook (9^{th} ed. 2002).

According to preferred embodiments of the present invention, compositions further comprising at least one wax are provided.

“Wax” means a lipophilic compound, solid at ambient temperature (25° C.), with a reversible solid/liquid change of state, having a melting point greater than or equal to 30° C.

The waxes used in accordance with the present invention can be any wax and is not particularly limited. The waxes can be hydrocarbon, fluorinated and/or silicone, and be of plant, mineral, animal and/or synthetic origin.

Suitable examples of waxes include, but are not limited to, Carnauba wax, candelilla wax, BIS-PEG-12 DIMETHICONE CANDELILLATE wax such as for example the Siliconyl Candelilla Wax marketed by the company KOSTER KEUNEN, hydrogenated Jojoba wax such as for example that marketed by the company DESERT WHALE, hydrogenated palm oil such as that marketed by the company SIO, rice bran wax, Sumac wax, ceresin waxes, laurel wax, Chinese insect wax, Shellac wax, hydrogenated olive oil such as Waxolive from the company SOLIANCE, waxes obtained by hydrogenation of olive oil esterified with C12 to C18 chain fatty alcohols such as those sold by the company SOPHIM under the brand names Phytowax Olive 12L44, 14L48, 16L55 and 18L57, waxes obtained by hydrogenation of castor oil esterified with cetyl or behenyl alcohol such as for example those which are sold under the names Phytowax Ricin 16 L 64 and Phytowax Ricin 22 L 73 by the company SOPHIM, hydrogenated Cameline wax, Ouricury wax, Montan wax, ozokerite waxes such as for example Wax SP 1020 P marketed by the company Strahl & Pitsch, microcrystalline waxes such as for example that sold under the brand name Microwax HW by the company PARAMELT, triglycerides of lauric, palmitic, cetylic and stearic acids (INCI name: hydrogenated coco glycerides) such as for example that sold under the brand name Softisan 100 by the company SASOL, polymethylene waxes such as for example that sold under the brand name Cirebelle 303 by the company SASOL, polyethylene waxes such as for example those sold under the brand names Performalene 400 polyethylene, Performalene 655 polyethylene and Performalene 500-L polyethylene by the company New Phase Technologies, alcohol-polyethylene waxes such as for example that marketed under the name Performacol 425 Alcohol by the company BARECO, the 95/5 ethylene/acrylic acid copolymer sold under the brand name AC 540 wax by the company Honeywell, hydroxyoctacosanyl hydroxy-stearate such as for example that sold under the brand name Elfacos C 26 by the company AKZO, octacosanyl stearate such as for example that marketed under the name Kester Wax K 82H by the company KOSTER KEUNEN, stearyl stearate such as for example that marketed under the name Liponate SS by the company LIPO CHEMICALS, pentaerythritol distearate such as for example that marketed under the name Cutina PES by the company COGNIS, the mixture of dibehenyl adipate, dioctadecyl adipate and di-eicosanyl adipate (INCI name C18-C22 dialkyl adipate), the mixture of dilauryl adipate and ditetradecyl adipate (INCI name: C12-C14 dialkyl adipate), the mixture of dioctadecyl sebacate, didocosyl sebacate and dieicosyl sebacate (INCI name: C18-C22 dialkyl sebacate) and the mixture of dioctadecyl octadecanedioate, didocosyl octanedioate and dieicosyl octanedioate (INCI name: C18-C22 dialkyl octanedioate) such as for example those marketed by the company COGNIS, pentaerythrityl tetrastearate such as for example Liponate PS-4 from the company Lipo Chemicals, tetracontanyl stearate such as for example Kester Wax K76H from the company KOSTER KEUNEN, stearyl benzoate such as for example Finsolv 116 from the company FINETEX, behenyl fumarate such as for example Marrix 222 from the company AKZO BERNEL, di-(trimethylol-1,1,1-propane) tetrastearate such as for example that which is offered under the name “HEST 2T-4S” by the company HETERENE, didotriacontanyl distearate such as for example Kester Wax K82D from the company KOSTER KEUNEN, polyethylene glycol montanate with 4 ethylene oxide units (PEG-4) such as for example that which is sold under the brand name Clariant Licowax KST1, hexanediol disalicylate such as for example Betawax RX-13750 marketed by the company CP Hall, dipentaerythritol hexastearate such as for example that which is sold under the brand name Hest 2P-6S by the company HETERENE, ditrimethylolpropane tetrabehenate such as for example that which is sold under the brand name Hest 2T-4B by the company HETERENE, Jojoba esters such as for example that which is sold under the brand name Floraester HIP by the company FLORATECH, mixtures of linear (C20-40) carboxylic acid/saturated hydrocarbons (INCI name: C20-40 acid polyethylene) such as for example Performacid 350 acid from the company NEW PHASE TECHNOLOGIES, synthetic wax of the Fischer-Tropsch type such as that marketed under the name Rosswax 100 by the company ROSS, cetyl alcohol, stearyl alcohol, behenyl alcohol, dioctadecyl carbonate such as for example Cutina KE 3737, saccharose polybehenate such as for example Crodaderm B from the company CRODA, and mixtures thereof, can in particular be cited. Such waxes are also described in U.S. patent application publication no. 2009/0142289, the entire contents of which is hereby incorporated by reference.

Waxes of plant origin such as carnauba wax, candelilla wax, hydrogenated jojoba wax, sumac wax, waxes obtained by hydrogenation of olive oil esterified with C12 to C18 chain fatty alcohols sold by the company SOPHIM in the Phytowax range (12L44, 14L48, 16L55 and 18L57), rice bran wax, cetyl, stearyl and behenyl alcohols, laurel wax and Ouricury wax are preferably used.

If present, the wax(es) are preferably present in a quantity of at least 5% by weight, preferably from about 5 to about 35% by weight, preferably from about 7.5 to about 30% by weight, and preferably from about 10 to about 25% by weight relative to the total weight of the composition, including all ranges and subranges therebetween.

According to preferred embodiments, the compositions of the present invention comprise at least one polyorganosiloxane copolymer. The polyorganosiloxane copolymer useful herein is preferably a polymer (homopolymer or copolymer) having at least one moiety which contains: at least one polyorganosiloxane group consisting of 1 to about 1000 organosiloxane units in the chain of the moiety or in the form of a graft, and at least two groups capable of establishing hydrogen interactions. Non-limiting examples of polyorganosiloxane copolymers are disclosed, for example in U.S. Pat. No. 8,945,525, the disclosure of which is hereby incorporated by reference in its entirety.

Additional polyorganosiloxane copolymers which may be used in the long-wear lip compositions of the present invention include those described in documents U.S. Pat. Nos. 5,874,069, 5,919,441, 6,051,216, and 5,981,680, the entire contents of which are hereby incorporated by reference in their entirety.

A preferred polyorganosiloxane copolymer for use in the present invention contain at least one moiety chosen from formula (III):

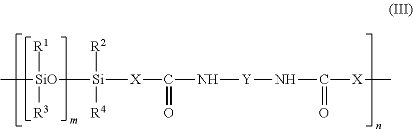

and formula (IV)

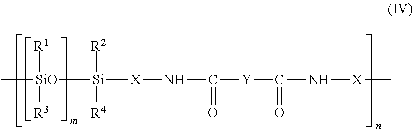

in which:

(a) R¹, R², R³ and R⁴ are the same or different and may be selected from the group consisting of methyl, ethyl, propyl, isopropyl, a siloxane chain, and phenyl;

(b) X is a linear or branched chain alkylene having 1-30 carbons;

(c) Y is selected from the group consisting of linear or branched chain alkylenes having 1-40 carbons;

(d) m is a number between 1 and 700;

(e) n is a number between 1 and 500.

Particularly preferred polyorganosiloxane copolymers useful herein are commercially available from Dow Corning under the tradenames DC 8178® and DC 8179®, which are known under the INCI denomination of Nylon-611/Dimethicone Copolymer.

If present, the polyorganosiloxane copolymer is preferably present in the compositions of the present invention in an amount ranging from about 1% to about 35% by weight, preferably from about 5% to about 25% by weight, and preferably from about 7% to about 15% by weight, including all ranges and subranges therebetween, all weights being based on the weight of the composition as a whole.

Suitable fillers and/or viscosity increasing agents include silicate clays such as, for example, silicate clays containing at least one cation which may be chosen from calcium, magnesium, aluminium, sodium, potassium, and lithium cations, and mixtures thereof. Non-limiting examples of such products include smectite clays such as montmorillonites, hectorites, bentonites, beidellites, saponites, vermiculites, stevensite, and chlorites. Preferred clays are synthetic silicate clays, most preferably lithium magnesium sodium silicate, commercially available from Rockwood under the tradename Laponite®. Other preferred examples of silicate clays which may be used in the present invention are chosen from lithium magnesium silicate, aluminum calcium sodium silicate, calcium magnesium silicate, sodium magnesium silicate, calcium aluminum borosilicate, magnesium aluminum silicate, sodium potassium aluminum silicate, and sodium silver aluminum silicate.

If present, the filler and/or viscosity increasing agent is preferably present in the compositions of the present invention in an amount of from 0.1% to 10% by weight, preferably in an amount of from 0.5% to 7%, preferably in an amount of from 1% to 5% by weight, based on the total weight of the composition.

According to preferred embodiments of the present invention, the compositions can further comprise a desired agent. The desired agent can be, for example, any colorant (pigment, dye, etc.), fiber or any additional film forming agent known in the art.

Acceptable colorants include pigments, dyes, such as liposoluble dyes, nacreous pigments, and pearling agents.

Representative liposoluble dyes which may be used according to the present invention include Sudan Red, DC Red 17, DC Green 6, ß-carotene, soybean oil, Sudan Brown, DC Yellow 11, DC Violet 2, DC Orange 5, annatto, and quinoline yellow.

Representative nacreous pigments include white nacreous pigments such as mica coated with titanium or with bismuth oxychloride, colored nacreous pigments such as titanium mica with iron oxides, titanium mica with ferric blue or chromium oxide, titanium mica with an organic pigment chosen from those mentioned above, and nacreous pigments based on bismuth oxychloride.

Representative pigments include white, colored, inorganic, organic, polymeric, nonpolymeric, coated and uncoated pigments. Representative examples of mineral pigments include titanium dioxide, optionally surface-treated, zirconium oxide, zinc oxide, cerium oxide, iron oxides, chromium oxides, manganese violet, ultramarine blue, chromium hydrate, and ferric blue. Representative examples of organic pigments include carbon black, pigments of D & C type, and lakes based on cochineal carmine, barium,

Suitable fibers include, but are not limited to, fibers which enable improvement of the lengthening effect. “Fiber” should be understood to mean an object of length L and diameter D such that L is much greater than D, D being the diameter of the circle in which the cross-section of the fibre is inscribed. In particular, the L/D ratio (or form factor) is selected in the band ranging from 3.5 to 2500, in particular from 5 to 500, and more particularly from 5 to 150. The fibers utilisable in the composition of the invention can be fibers of synthetic or natural origin, mineral or organic. They can be short or long, unitary or structured, for example, braided, hollow or full. They can be of any shape and in particular of circular or polygonal cross-section (square, hexagonal or octagonal) depending on the specific application envisaged. In particular, their ends are blunted and/or polished to avoid injury. They can be rigid or non-rigid fibers. They can be of synthetic or natural origin, mineral or organic. They can be surface treated or not, coated or not, and colored or not.

If present, the desired agent is preferably present in the compositions of the present invention in an amount of from 0.1% to 30% by weight, preferably in an amount of from 0.5% to 25%, preferably in an amount of from 1% to 20% by weight, based on the total weight of the composition.

According to other preferred embodiments, methods of treating, caring for and/or enhancing the appearance of hair, eyebrows or eyelashes by applying compositions of the present invention to the hair, eyebrows or eyelashes in an amount sufficient to treat, care for and/or enhance the appearance of the hair, eyebrows or eyelashes are provided. In accordance with these preceding preferred embodiments, the compositions of the present invention are applied topically to the desired area of the hair, eyebrows or eyelashes in an amount sufficient to treat, care for and/or enhance the appearance of the hair, eyebrows or eyelashes. The compositions may be applied to the desired area as needed, preferably once or twice daily, more preferably once daily and then preferably allowed to dry before subjecting to contact such as with clothing or other objects (for example, a topcoat). Preferably, the composition is allowed to dry for about 1 minute or less, more preferably for about 45 seconds or less. The composition is preferably applied to the desired area that is dry or has been dried prior to application, or to which a basecoat has been previously applied.

According to a preferred embodiment of the present invention, compositions having improved cosmetic properties such as, for example, increased removability from hair, eyebrows or eyelashes to which they have been applied are provided. Preferably, the composition is a composition for eyelashes. Most preferably, the composition is a mascara composition further comprising at least one colorant.

According to a preferred embodiment of the present invention, methods of improving the removal properties (from hair, eyebrows and/or eyelashes) of a water-in-oil (w/o) emulsion composition comprising at least one siloxysilicate resin, and optionally at least one wax, by adding at least one latex comprising polymer comprising at least one styrene group in an amount sufficient to add from about 2% to about 6% by weight with respect to the total weight of the composition of the polymer comprising at least one styrene group to the emulsion composition, resulting in increased removal properties of the emulsion composition, are provided. Preferably, the composition is a composition for eyelashes. Most preferably, the composition is a mascara composition further comprising at least one colorant.

According to a preferred embodiment of the present invention, methods of making a water-in-oil (w/o) emulsion composition for hair, eyelashes and/or eyebrows comprising combining at least one latex comprising polymer comprising at least one styrene group in an amount sufficient to add from about 2% to about 6% by weight with respect to the total weight of the composition of the polymer comprising at least one styrene group and at least one siloxysilicate resin, and optionally at least one wax, to form the emulsion composition are provided. Preferably, the composition is a composition for eyelashes. Most preferably, the composition is a mascara composition further comprising at least one colorant.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective measurements. The following examples are intended to illustrate the invention without limiting the scope as a result. The percentages are given on a weight basis.

### EXAMPLES

### Example 1—Sample Mascara Composition

| Phase | Chemical Name | % wt/wt |
|---|---|---|
| A | Surfactants | 5 |
| A | Waxes | 15 |
| A | Oils | 25 |
| A | Preservatives | 1 |
| A | Colorants -- Iron Oxides | 8 |
| A | Water | QS |
| A | Polyol | 2 |
| A | Trimethylsiloxsilicate | 8 |
| A | Clays | 3 |
| B | Styrene/acrylates/ammonium | 4 |
| | methacrylate copolymer | |

Total %100.00

Sample Procedure for Preparing Compositions of the Present Invention:

In metal container A, All phase A materials were added and heated until 90° C.

When metal container A was at 90° C., homogenized for 30 minutes at 2000 RPM.

After the mixture was uniform and cooled to 45° C., added B subsequently, and mixed for 10 mins.

Batch Cooled to 30° C.

### Example 2—Determination of Removal Properties

Water-in-oil emulsions containing the same base ingredients but varying amounts of (1) latex containing styrene/acrylates/ammonium methacrylate copolymer (ranging from 0% to 10%); (2) MQ resin in combination with nylon 611/dimethicone (ranging from 0% to 16.7%); and (3) waxes (ranging from 17% to 25.5%) were prepared and tested for removal properties as follows:

Compositions were applied to artificial eyelashes and allowed to dry at room temperature. Once dried, the compositions were subjected to contact with removal solvent (makeup remover) using a cotton pad until the composition was completely removed from the eyelashes. The number of strokes it took to completely remove compositions from eyelashes was counted and recorded, with 4=easy removal; 5=good removal; 6=poor removal; and 7=difficult removal.

It was found that compositions containing MQ resin, and optionally at least one wax, containing from about 2% to about 6% styrene/acrylates/ammonium methacrylate copolymer possessed easy or good removal properties, whereas compositions containing the copolymer in amounts outside of the 2%-6% range possessed poor or difficult removal properties. It was also found that removal properties were generally not affected by the amount of MQ resin present in the compositions.

1. A water-in-oil emulsion composition comprising (a) water, (b) at least one oil, and (c) a film-forming component comprising (i) at least one latex comprising polymer comprising at least one styrene group, wherein the composition comprises from about 2% to about 6% by weight with respect to the total weight of the composition of the polymer comprising at least one styrene group, and (ii) at least one siloxysilicate resin, wherein the composition is in the form of a composition for eyelashes.

2. The composition of claim 1, wherein the polymer comprising at least one styrene group is a copolymer comprising (1) styrene; (2) ammonium (meth)acrylates, and (3) (meth)acrylate and/or (meth)acrylic groups.

3. The composition of claim 1, wherein the polymer comprising at least one styrene group is a styrene/acrylates/ammonium methacrylate copolymer.

4. The composition of claim 1, wherein the siloxysilicate resin is a trimethylsiloxysilicate resin.

5. The composition of claim 1, further comprising at least one wax in an amount from about 10% to about 25% by weight of the total weight of the composition.

6. The composition of claim 1, further comprising at least one colorant.

7. The composition of claim 1, further comprising at least one silicate clay.

8. The composition of claim 1, wherein the siloxysilicate resin is present in an amount from about 1% to about 10% by weight of the total weight of the composition.

9. The composition of claim 1, wherein the weight ratio of siloxysilicate resin to polymer comprising at least one styrene group present ranges from 1:1 to about 5:1.

10. The composition of claim 1, wherein the weight ratio of siloxysilicate resin to polymer comprising at least one styrene group present ranges from 2:1 to about 4:1.

11. The composition of claim 1, in the form of a mascara.

12. A method of making up eyelashes comprising applying the composition of claim 1 to eyelashes.

13. A method of improving removal properties from eyebrows and/or eyelashes of a water-in-oil emulsion composition comprising a film-forming component comprising at least one siloxysilicate resin, and optionally at least one wax, comprising adding to the film-forming component of the composition at least one latex comprising polymer comprising at least one styrene group in an amount sufficient to add from about 2% to about 6% by weight with respect to the total weight of the composition of the polymer comprising at least one styrene group.

14. A water-in-oil emulsion composition comprising:
(a) water,
(b) at least one oil,
(c) at least one wax in an amount from about 10% to about 25% by weight of the total weight of the composition,
(d) a film-forming component comprising (i) at least one latex comprising polymer comprising at least one styrene group, wherein the composition comprises from about 2% to about 6% by weight with respect to the total weight of the composition of the polymer comprising at least one styrene group, and (ii) at least one trimethylsiloxysilicate resin,
wherein the composition is in the form of a mascara.

15. The composition of claim 1, wherein the film-forming component contains greater than 50% by weight of the polymer comprising at least one styrene group and the siloxysilicate resin, the percentage being based on their total weight with respect to the total weight of the film-forming component.

16. The method of claim 13, wherein the film-forming component contains greater than 50% by weight of the polymer comprising at least one styrene group and the siloxysilicate resin, the percentage being based on their total weight with respect to the total weight of the film-forming component.

17. The composition of claim 14, wherein the film-forming component contains greater than 50% by weight of the polymer comprising at least one styrene group and the siloxysilicate resin, the percentage being based on their total weight with respect to the total weight of the film-forming component.

18. The composition of claim 1, wherein the siloxysilicate resin is present in an amount from about 1% to about 10% by weight of the total weight of the composition and the weight ratio of siloxysilicate resin to polymer comprising at least one styrene group present ranges from 1:1 to about 5:1.

19. The composition of claim 15, wherein the siloxysilicate resin is present in an amount from about 1% to about 10% by weight of the total weight of the composition and the weight ratio of siloxysilicate resin to polymer comprising at least one styrene group present ranges from 1:1 to about 5:1.
